(19) United States
(12) Patent Application Publication
(10) Pub. No.: US 2026/0327703 A1
(43) Pub. Date: Oct. 22, 2026

(54) USE OF MICRORNAS AS TOOLS FOR THE DIAGNOSIS AND DIFFERENTIATION OF TAUOPATHIES

(71) Applicants: UNIVERSITA’ DI TRENTO, TRENTO (IT); ISTITUTO SUPERIORE DI SANITA’, ROMA (IT); UNIVERSITÁ DEGLI STUDI DI ROMA “LA SAPIENZA", ROMA (IT)

(72) Inventors: Michela Alessandra DENTI, TRENTO (IT); Leonardo RICCI, TRENTO (IT); Margherita GRASSO, ROMA (IT); Andrea ZENI, TRENTO (IT); Paola PISCOPO, ROMA (IT); Roberto RIVABENE, ROMA (IT); Giuseppe BRUNO, ROMA (IT)

(21) Appl. No.: 19/481,104

(22) PCT Filed: Apr. 24, 2024
(86) PCT No.: PCT/IT2024/050079

§ 371 Date: Nov. 3, 2025

(30) Foreign Application Priority Data
May 4, 2023 (IT) 102023000008841

(51) Int. Cl. C12Q 1/6883; C12N 15/113

(52) U.S. Cl. CPC C12Q 1/6883; C12N 15/113; C12N 2310/141; C12Q 2600/178

(57) ABSTRACT

The present invention relates to microRNAs for use in diagnostic methods for diagnosing and/or for distinguishing by diagnosis Alzheimer's disease (AD), Frontotemporal Dementia (FTD), and possibly other tauopathies, and/or for measuring therapeutic effect of a treatment of FTD or AD.

### TECHNICAL FIELD

The present invention relates to the field of microRNAs (abbreviated miRNAs) for use in diagnostic methods and/or therapeutic methods for treatment of Alzheimer's disease, Frontotemporal Dementia (FTD), or other tauopathies.

### BACKGROUND OF THE INVENTION

Dementia is one of the most common diseases in elderly people and hundreds of thousand new cases per year of Alzheimer's disease (AD) are estimated. While the recent decade has seen significant advances in the development of novel biomarkers to identify dementias at their early stage, a great effort has been recently made to identify biomarkers able to improve differential diagnosis. However, only few potential candidates, mainly detectable in cerebrospinal fluid (CSF), have been described so far.

The World Health Organization declared that in 2019 over 50 million people in the world have some form of dementia, a condition of chronic and progressive brain function disruption leading to a decline of the cognitive faculties of the person. There are many causes for dementia, including primary neurologic, medical and neuropsychiatric conditions. Among those primary neurodegenerative forms, Frontotemporal Dementia (FTD) and Alzheimer's disease (AD) share similar clinical symptoms. This fact can often cause misdiagnoses and, consequently, result in inappropriate treatments.

On the other hand, there is an urgent and unmet need of non-invasive and reliable biomarkers to identify patients during early, asymptomatic stages of the disease, when a pharmacological intervention is still possible.

Recently, a novel and promising class of biomarkers for diagnosing and prognosing central nervous system (CNS) diseases has been proposed. Several studies have shown that alterations of the microRNAs (miRNAs), essential components of gene-regulatory networks, play a critical role in the occurrence and development of a variety of neurodegenerative diseases. MiRNAs are a class of small non-coding RNAs, which mainly act as powerful post-transcriptional regulators. In particular, miRNAs target specific mRNAs inducing the repression of translation or their degradation to modulate and fine-tune gene expression levels. They undergo a specific tissutal and temporal distribution, as indicated by several studies on miRNA profiling in different chronic diseases. The alteration of their expression has been associated with almost all cancers and many non-cancer diseases such as AD, multiple sclerosis and heart failure. MiRNAs result long lasting in all human biofluids, because their resistance to degradation. In fact, circulating miRNAs are transported by extracellular vesicles or lipoproteins, and their complexation with proteins, included in the RNA-induced silencing complexes (RISC) protects them from enzymatic degradation.

Of the about 2,500 mature miRNAs identified in humans, 70% were estimated to be expressed in the nervous system, although only a handful of them are expressed in a brain specific or brain-enriched manner. MiRNAs expressed in the CNS mainly regulate neural differentiation, synaptic plasticity and neurite outgrowth.

MiRNAs are easily detectable in body fluids (plasma, serum, urine, saliva, milk, CSF), using specific simple and sensitive assays. This fact, together with their stability even after a long time period of storage, makes miRNAs good candidates as possible diagnostic and/or prognostic biomarkers in different pathophysiological processes for neurodegenerative disorders and conditions affecting the CNS, particularly in older adults.

Tau is encoded by the MAPT (microtubules associated protein Tau) gene. It interacts with microtubules, modulating their dynamic instability and axonal transport, and affects synaptic function and plasticity. Tau phosphorylation regulates the activity of the proteins and it is directly implicated in pathological processes. In fact, phosphorylation is able to reduce the Tau tendency to bind microtubules, augmenting its unbounded amount that tend to aggregate. Tau has a relevant role in both AD and FTD. Elevated phosphorylation and aggregation of Tau are widely considered pathological hallmarks in AD, while filamentous inclusions of Tau represent a feature in cases of inherited and sporadic FTDs. Moreover, several MAPT mutations were associated to FTD, especially those that alter the ratio between the 6 different Tau isoforms, favouring the more aggregation-prone forms.

### SUMMARY OF THE INVENTION

The objective of the present invention is to provide an effective diagnostic method, detecting specific miRNAs used as molecular biomarkers to diagnose and/or differentiate AD and FTDs as other tauopathies. For this propose, a search for miRNAs regulating MAPT translation has been carried out. Since, given a mRNA, in silico tools to predict miRNA interactors are still not reliable and yield a vast number of candidates, a capture technology able to find the miRNAs directly bound to the MAPT transcript in cell lines was used. Afterwards, the identified miRNAs were analyzed in human samples from FTD and AD patients and in matched healthy controls.

Further characteristics and advantages of the use of the miRNAs of invention, and their diagnostic and therapeutical use, will result from the description of the examples of the invention, provided as an indication of the invention.

### BRIEF DESCRIPTION OF THE DRAWINGS

FIG. 1.

MAPT mRNA miR-CATCH. (A) Schematic representation of miR-CATCH 1) active mRNA:miRISC complexes are cross-linked using formaldehyde fixation; 2) cells are lysed; 3) capture oligonucleotide probes complexed with metal beads are hybridized to the target sequence in the mRNA of interest; 4) target mRNAs and the miRISC complexes bound to them are pulled down using magnetic separation; 5) the unbound non-target mRNAs (in grey) are washed away resulting in the enrichment of target mRNA:miRISCs complexes; 6) cross-links are reversed and capture oligonucleotides removed enabling measurement of the enrichment of the target MAPT mRNA and of the captured miRNAs (compared to a scramble capture oligo). (B) In the red call-out, the accessible, partially single-stranded region chosen as target for the capture oligonucleotide is shown. The region is located between bases 1855 and 1875 of transcript variant 9 and is present in all variants. (C) miR-CATCH was performed in Kelly cells using capture oligonucleotide (Capture; n=3) or scrambled control oligonucleotide (Scrambled; n=3). (D) Identification of MAPT mRNA-binding miRNAs. Heat Map showing the miRNAs significantly enriched in MAPT-miR-CATCHed samples. Data are reported as Fold Change (FC=2-DeltaCt). The ten miRNAs selected for further validation are shown in bold. (E) Enrichment of miR-190b, miR-146a-5p, let7a-5p, let7e-5p, miR-320b, miR-320a, miR-320c, miR-320d, miR-92a-3p, and miR-1260a. **** p<0.0001.

FIG. 2.

miRNA overexpression and inhibition effects on Tau levels in Kelly cells. A Effects of the overexpression of miR-92a-3p, miR-320a, miR-320b, miR-320c, miR-320d, miR-1260a, miR-190b, let7a-5p, and let7e-5p on Tau protein levels in Kelly cells. Densitometric analysis from three independent experiments is shown in the lower panel. Data are shown as mean±S.D. and are compared by t-test for negative control miR (C: Control) versus each miR of interest (p-value: *P<0.05; ****P<0.0001 (B) Effects of the inhibition of miR-320a, miR-320b and miR-92a-3p on Tau protein levels in Kelly cells. Densitometric analysis from three independent experiments is shown in the lower panel. Data are shown as mean±S.D. and are compared by t-test for LNA inhibitor negative control (Control LNA) versus each miR inhibitor of interest (p-value: *P<0.05; **P<0.01; ****P<0.0001). (C) Predicted binding sites of miR-320a, miR-320b and miR-92a-3p on MAPT mRNA. The miRNAs' target sequences in MAPT mRNA and their hybridization minimum free energy (mfe) were computed with RNAHybrid. CDS: Coding Sequence; 3′UTR: 3′ Untranslated Region.

FIG. 3.

Scatter plots of miRNAs expression levels (2-ACt) among CT, FTD and AD in the whole population and in males and females.

The data are represented as the mean±SEM. CT=Controls; FTD=Frontotemporal Dementia; AD=Alzheimer's Disease; *p≤0.05; **p≤0.01; ***p≤0.001.

FIG. 4.

Receiver operating characteristic curve of differentially expressed miR-92a-3p, miR-320a and miR-320b for CT versus FTD, CT versus AD and FTD versus AD in the whole population and in males and females. CT=Controls; FTD=Frontotemporal Dementia; AD=Alzheimer's Disease; AUC=Area Under Curve.

FIG. 5.

Table 1 Characterization of studied population. All values are reported as mean±standard deviation. bvFTD=behavioural variant FTD; svFTD=semantic variant FTD; SD=Standard Deviation.

FIG. 6.

ROC curves (top), histograms (middle) and score plot (bottom) of the combinations of ΔCt of miR-92a, miR-320a and miR-320b, for samples belonging to the target class AD (blue) and class HCs (red). In the histograms, overlapping regions are in magenta. The x-axis represents the score. Each histogram is normalized to the respective set size and thus corresponds to a sample density. The bold lines represent the Gaussian probability densities that fit the data. In the score plot, the bold black lines represents the threshold line for diagnosis while the black dashed lines represent the threshold plus/minus one standard deviation.

FIG. 7.

ROC curves (top), histograms (middle) and score plot (bottom) of the combinations of ΔCt of miR-92a, miR-320a and miR-320b, for samples belonging to the target class FTD (blue) and class HCs (red). In the histograms, overlapping regions are in magenta. The x-axis represents the score. Each histogram is normalized to the respective set size and thus corresponds to a sample density. The bold lines represent the Gaussian probability densities that fit the data. In the score plot, the bold black lines represents the threshold line for diagnosis while the black dashed lines represent the threshold plus/minus one standard deviation.

FIG. 8.

ROC curves (top), histograms (middle) and score plot (bottom) of the combinations of ΔCt of miR-92a, miR-320a and miR-320b, for samples belonging to the target class FTD (blue) and class AD (red). In the histograms, overlapping regions are in magenta. The x-axis represents the score. Each histogram is normalized to the respective set size and thus corresponds to a sample density. The bold lines represent the Gaussian probability densities that fit the data. In the score plot, the bold black lines represents the threshold line for diagnosis while the black dashed lines represent the threshold plus/minus one standard deviation.

### DETAILED DESCRIPTION OF THE INVENTION

An object of the present invention is an in vitro diagnostic method for diagnosing and/or for distinguishing by diagnosis Alzheimer's disease (AD) and Frontotemporal Dementia (FTD), and/or for measuring therapeutic effect of a treatment of FTD or AD, comprising the quantitative analysis of at least one RNA sequence selected among the group consisting of RNA Seq. ID_1, RNA Seq ID._2 and RNA Seq. ID_3, or a combination thereof, or a combination of at least one RNA sequence selected among the group consisting of RNA Seq. ID_1, RNA Seq ID_2 and RNA Seq. ID_3 with at least another biomarker, in a human fluid.

RNA Seq ID_1:
miR-320b
Sequence:
AAAAGCUGGGUUGAGAGGGCAA.
RNA Seq. ID_2:
miR-320a
Sequence:
AAAAGCUGGGUUGAGAGGGCGA.
RNA Seq. ID_3:
miR-92a-3p
Sequence:
UAUUGCACUUGUCCCGGCCUGU.

Indeed, it has been surprisingly found that exactly the those three specific RNA sequences are well suitable for diagnosing and/or for distinguishing by diagnosis Alzheimer's disease (AD) and Frontotemporal Dementia (FTD), and/or for measuring therapeutic effect of a treatment of FTD or AD.

A human fluid can be blood and derivatives thereof such as plasma, serum, vesicles, extracellular derivate, or, a human fluid can be, for instance, cerebrospinal fluid sample.

The quantitative analysis of the miRNAs in the in vitro diagnostic method can be performed according to the known methods for this kind of analysis, among the others, preferably, the quantitative analysis can be performed by PCR.

The in vitro diagnostic method can be carried out according to a method comprising the following steps:
a) blood sample collection,
b) isolation of plasma or serum from blood,
c) optionally, RNA extraction,
d) detection of at least one RNA sequence selected among the group consisting of Seq. ID_1, RNA Seq. ID._2 and RNA Seq. ID_3.

The step c) is optional, but RNA extraction step is preferred.

According to a preferred embodiment of the in vitro diagnostic method, the RNA sequence is RNA Seq. ID_1.

According to a preferred embodiment, the in vitro diagnostic method comprises the quantitative analysis of RNA sequence being RNA Seq. ID_2.

According to a preferred embodiment, the in vitro diagnostic method comprises the quantitative analysis of RNA sequence being RNA Seq. ID_3.

According to a more preferred embodiment, the in vitro diagnostic method comprises the quantitative analysis of two RNA sequences being RNA Seq ID_2 and RNA Seq. ID_3.

According to a more preferred embodiment, the in vitro diagnostic method comprises the quantitative analysis of three RNA sequences being RNA Seq. ID_1, RNA Seq ID_2 and RNA Seq. ID_3.

An other object is a method for selecting subjects for enrollment in a clinical trial involving treatment of FTD or AD, the method comprising: determining by diagnosis according to the method described above, the subject as being suitable for clinical trial.

An other object is a diagnostic device or diagnostic kit configured for carrying out quantitative analysis of at least one RNA sequence selected among the group consisting of RNA Seq. ID_1, RNA Seq ID_2 and RNA Seq. ID_3, or a combination thereof, or a combination of at least one RNA sequence selected among the group consisting of RNA Seq. ID_1, RNA Seq ID_2 and RNA Seq. ID_3 with at least another biomarker, in a human fluid,

wherein said diagnostic device or diagnostic kit comprises a toolkit configured for identify and quantify at least one RNA sequence selected among the group consisting of RNA Seq. ID_1, RNA Seq ID_2 and RNA Seq. ID_3, or a combination thereof, or a combination of at least one RNA sequence selected among the group consisting of RNA Seq. ID_1, RNA Seq. ID_2 and RNA Seq. ID_3 with at least another biomarker, in a human fluid.

The above said toolkit can be a PCR toolkit, or a toolkit based on next-generation sequencing or based on nucleic acids sensors.

An other object is a RNA sequence selected among the group consisting of Seq. ID_1, RNA Seq. ID_2 and RNA Seq. ID_3, or a combination thereof, or a combination of at least one RNA sequence selected among the group consisting of RNA Seq. ID_1, RNA Seq. ID_2 and RNA Seq. ID_3 with at least another biomarker, for use in diagnostic method, wherein said diagnostic method diagnoses and/or distinguishes by analysis Alzheimer's disease (AD), Frontotemporal Dementia (FTD), or wherein said diagnostic method measures therapeutic effect of a treatment of FTD or AD.

According to a preferred embodiment of said use, the diagnostic method comprises the quantitative analysis of at least one RNA sequence selected among the group consisting of RNA Seq. ID_1, RNA Seq. ID_2 and RNA Seq. ID_3, or a combination thereof, or a combination of at least one RNA sequence selected among the group consisting of RNA Seq. ID_1, RNA Seq. ID_2 and RNA Seq. ID_3 with at least another biomarker, in a human fluid.

According to a preferred embodiment of said use, the diagnostic method is carried by measuring the concentration of at least one RNA sequence selected among the group consisting of seq. ID_1, RNA Seq. ID_2 and RNA Seq. ID_3 in a human fluid.

According to a preferred embodiment of said use, the human fluid is blood or derivatives thereof.

According to a preferred embodiment of said use, the RNA sequence is RNA Seq. ID_1.

According to a preferred embodiment of said use, the diagnostic method comprises the quantitative analysis of RNA sequence being RNA Seq. ID_2.

According to a preferred embodiment of said use, the diagnostic method comprises the quantitative analysis of RNA sequence being RNA Seq. ID_3.

According to a preferred embodiment of said use, the diagnostic method comprises the quantitative analysis of two RNA sequences being RNA Seq. ID_2, and RNA Seq. ID_3.

According to a preferred embodiment of said use, the diagnostic method comprises the quantitative analysis of three RNA sequences being RNA Seq. ID_1, RNA Seq. ID_2 and RNA Seq. ID_3.

Another object is a RNA sequence selected among the group consisting of seq. ID_1, RNA Seq. ID_2 and RNA Seq. ID_3, or a combination thereof, or a combination of at least one RNA sequence selected among the group consisting of RNA Seq. ID_1, RNA Seq. ID_2 and RNA Seq. ID_3 with at least another biomarker for use in a method of treatment of Alzheimer's disease, Frontotemporal Dementia (FTD), or other tauopathies.

According to a preferred embodiment of said use, the method of treatment comprises administrating a therapeutically effective amount of said RNA sequence.

An object is a pharmaceutical composition comprising at least an RNA sequence selected among the group consisting of seq. ID_1, RNA Seq. ID_2 and RNA Seq. ID_3, or a combination thereof, for use in a method of treatment of Alzheimer's disease, Frontotemporal Dementia (FTD), or other tauopathies.

Said pharmaceutical composition, optionally, further comprises at least an other therapeutical compound or active pharmaceutical compound.

An other object is an oligonucleotide whose sequence, or part thereof, is complementary to part or entire RNA seq. ID_1, RNA Seq ID_2 or RNA Seq. ID_3, for use as inhibitor of seq. ID_1, RNA Seq. ID_2 or RNA Seq. ID_3 in a method of treatment of Alzheimer's disease, Frontotemporal Dementia (FTD), or other tauopathies.

An other object is an oligonucleotide whose sequence, or part thereof, is complementary to part of or the entire target sequence of RNA seq. ID_1, RNA Seq ID_2 or RNA Seq. ID_3 on MAPT mRNA, for use as inhibitor of the interaction of seq. ID_1, RNA Seq. ID_2 or RNA Seq. ID_3 with MAPT mRNA, in a method of treatment of Alzheimer's disease, Frontotemporal Dementia (FTD), or other tauopathies.

Method: miRNAs regulating MAPT translation were searched. A capture technology able to find the miRNAs directly bound to the MAPT transcript in cell lines was used. Afterwards the levels of these miRNAs were evaluated in plasma samples from FTD (n=42) and AD patients (n=33) and relative healthy controls (HCs) (n=42) by using qRT-PCR.

Results: Firstly, 29 miRNAs that interact with the MAPT transcript were found. Ten miRNAs have been selected to verify their effect on Tau levels increasing or reducing miRNA levels by using cell transfections with plasmids expressing the miRNAs genes or LNA antagomiRs, respectively. Following the results obtained, miR-92a-3p, miR-320a and miR-320b were selected to analyse their levels in plasma samples of patients with FTD and AD respect to HCs. The analysis showed that the miR-92a-1-3p was under-expressed in both AD and FTD compared to HCs. Moreover, miR-320a was upregulated in FTD vs AD patients, particularly in men when we stratified by sex. Respect to HC, the only difference is showed in men with AD, who have reduced levels of this miRNA. Instead, miR-320b is up-regulated in both dementias, but only patients with FTD maintain this trend in both genders. A combination of the measurements of the three miRNAs performs better as a biomarker for the differential diagnosis of AD and FTD.

### 1. MiR-CATCH Identifies miRNAs Targeting MAPT mRNA

With the aim of exploring circulating miRNAs to be used as possible biomarkers in FTD and AD differential diagnosis, we decided to look for miRNAs with a functional relevance in these neurodegenerative diseases. We reasoned that Tau protein expression and accumulation has a role in both dementias, and we set out to find which miRNAs directly regulate MAPT expression.

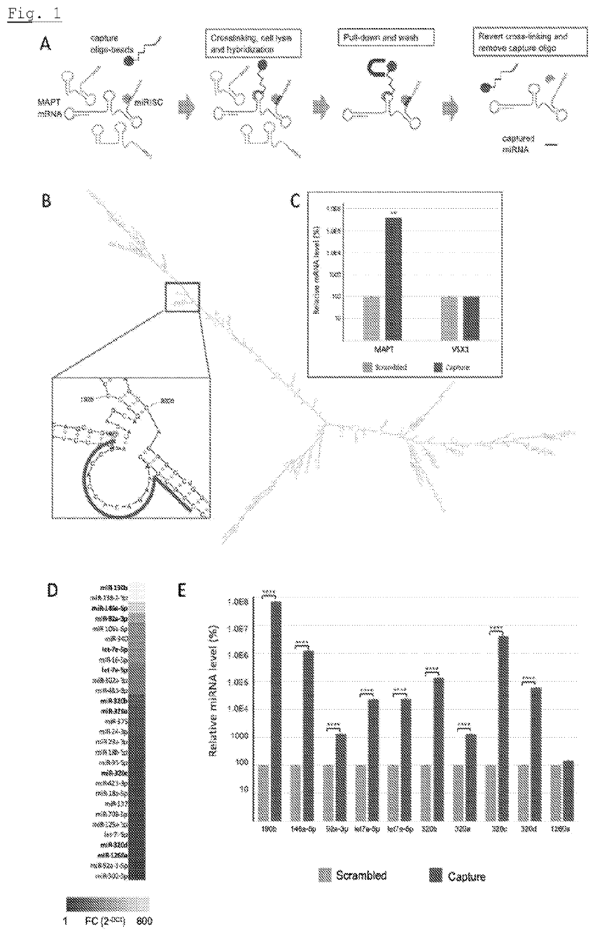

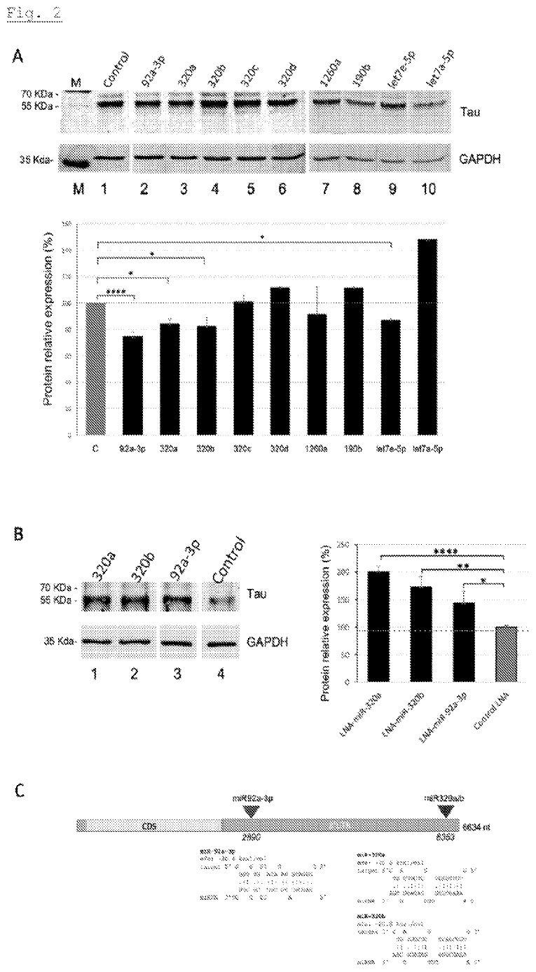

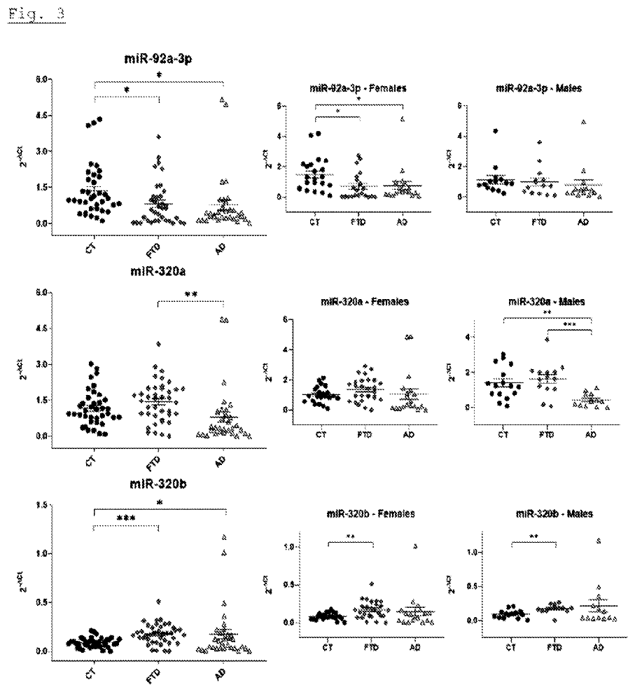

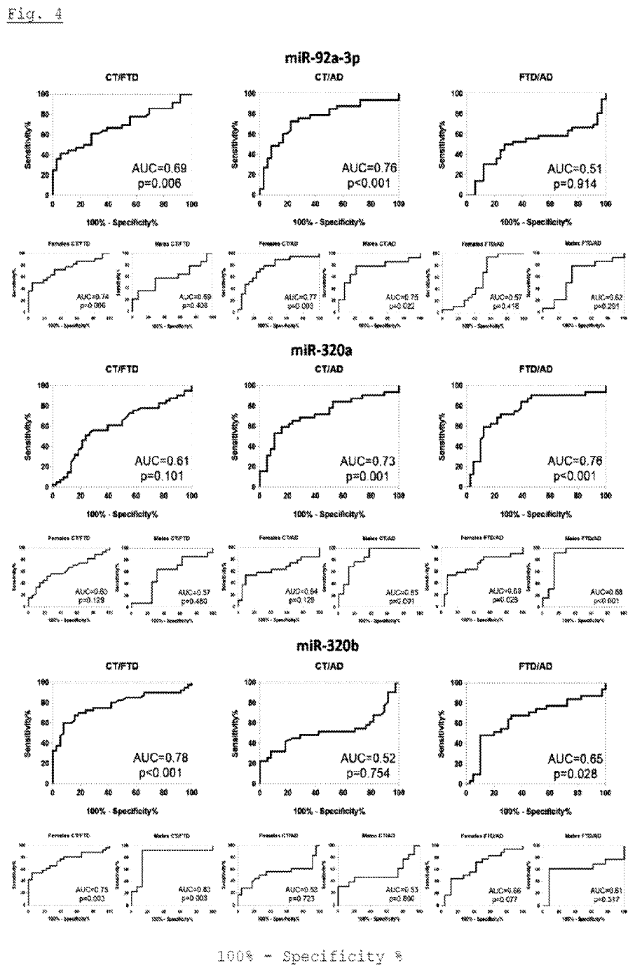

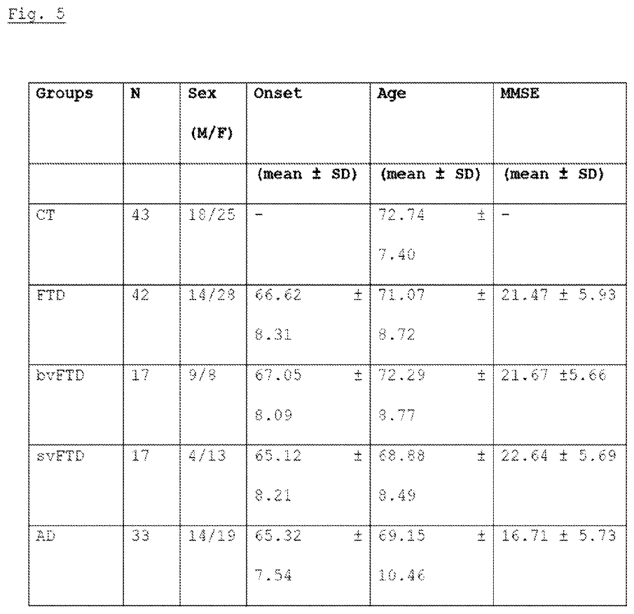

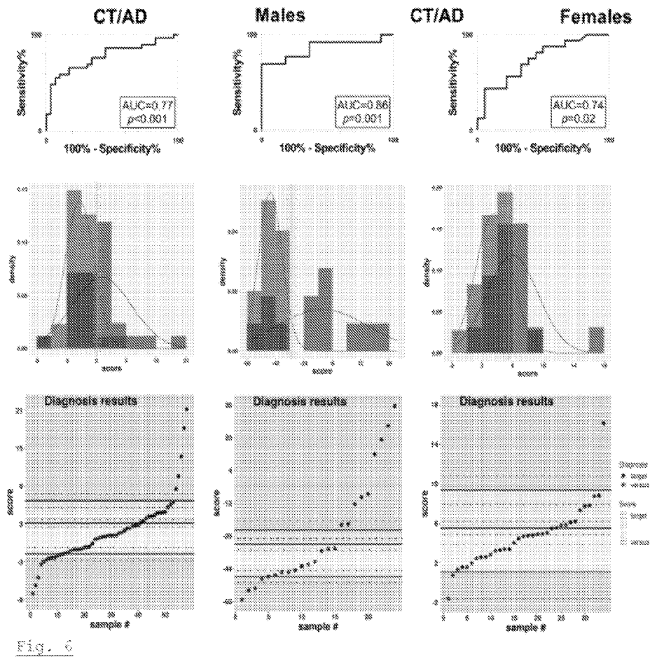

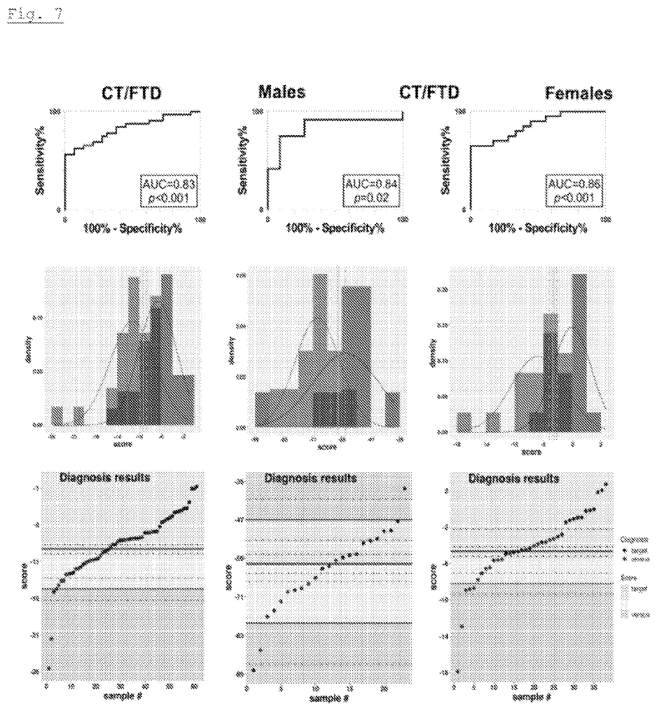

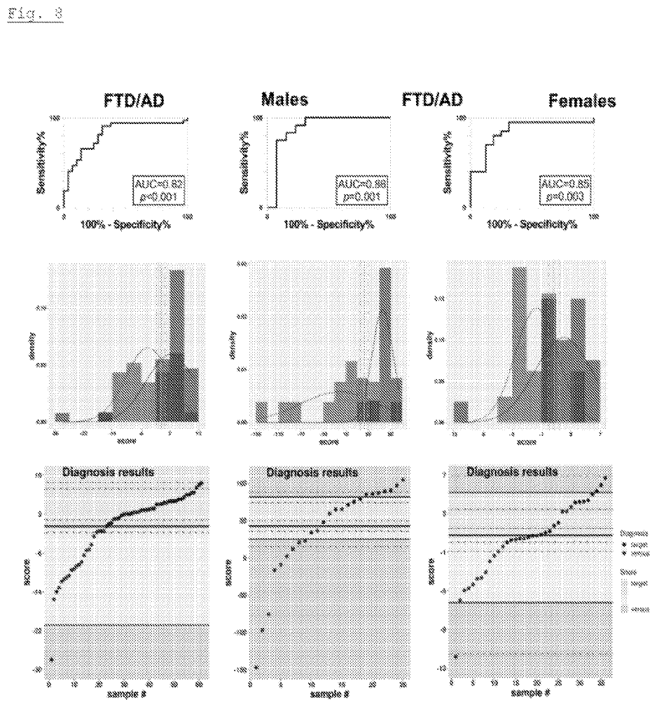

The use of computational analysis to accurately predict putative miRNAs that regulate specific mRNAs remains a challenging process, due to the high false-positive rates of predictions and the use of different algorithms that yield heterogeneous results. To identify the miRNAs binding to the MAPT mRNA, we therefore used an approach called MicroRNA Capture Affinity Technology (miR-CATCH, FIG. 1A) (Hassan T, et al., Nucleic Acids Res (2013); 41:6.

The method relies on the reversible crosslinking of active mRNA:miRISC complexes in the cells, using formaldehyde. Subsequently, cells are lysed and capture oligonucleotide probes complexed with magnetic beads are hybridized to the target sequence in the mRNA of interest. Target mRNAs and the associated miRISC complexes are then pulled down using magnetic separation and the unbound non-target mRNAs are washed away. Finally, crosslinks are reversed and capture oligonucleotides removed, enabling measurement of the enrichment of the target MAPT mRNA and of the captured miRNAs (compared to a scramble capture oligo).

To design a MAPT-specific capture oligonucleotide, the secondary structure of MAPT mRNA was predicted in silico and scanned for single stranded regions (FIG. 1B). MAPT mRNA exists in several variants resulting from alternative splicing of exons 2, 3 and 10. As we aimed at pulling down all isoforms, we modelled the longest variant (variant 9 MAPT mRNA, NCBI NM_001377265.1) and chose a target region present in all isoforms. The DNA capture oligonucleotide was designed to bind region 1855-1875 of MAPT mRNA and synthesised to carry a biotin at its 5′end. A scramble 5′-biotinylated oligonucleotide was also designed, to be used as a negative control.

Cells were crosslinked by formaldehyde and subsequently lysed and processed with the biotinylated capture oligonucleotide, in order to isolate MAPT mRNA:miRNA complexes by using streptavidin-coated magnetic beads. In parallel, cells were processed with the scramble biotinylated oligonucleotide. After crosslinking reversion, the eluted samples were analysed by qRT-PCR, which showed about 4000-fold enrichment of MAPT mRNA in samples processed with the capture oligonucleotide (“capture”), compared to those processed with the scrambled control oligonucleotide (“scramble”) (FIG. 1C).

As the capture 20-mer oligonucleotide showed 17-nt complementarity to the VSX1 mRNA in a BLAST analysis (see Materials and Methods), we performed qRT-PCR analyses also with primers specific for the VSX1 mRNA, a transcript not related to MAPT mRNA. As shown in FIG. 1C, VSX1 mRNA was not enriched in the capture samples, compared to scramble samples.

Samples were further analysed for the presence of 752 different miRNAs by miRNome qRT-PCR Human panel I+II (Exiqon). Data are available at NCBI's Gene Expression Omnibus (GEO) (https://www.ncbi.nlm.nih.gov/geo) under accession number GSE225256. As shown in FIG. 1D and detailed in Supplementary Table 2, 29 miRNAs were significantly enriched in capture samples compared to scramble samples. Out of these, 10 were selected for validation: let-7e-5p, let-7a-5p, miR-92a-3p, miR-146a-5p, miR-190b, miR-320a, miR-320b, miR-320c, miR-320d, miR-1260a (in bold in FIG. 1D).

The relative enrichment of 9 of the selected miRNAs in the capture samples compared to scramble samples was confirmed by qRT-PCR TaqMan single assays (FIG. 1E). In descending order of enrichment these miRNAs were: miR-190b (6.7×105-fold), miR-320c (4×104-fold), miR-146a-5p (1.2×104-fold), miR-320b (1.3×103-fold), miR-320d (5.8×102-fold), let-7e-5p (2.3×102-fold), let-7a-5p (2.3×102-fold), miR-320a (1.3×101-fold), miR-92a-3p (1.3×101-fold). The enrichment of miR-1260a was not significant when assessed with the TaaMan single assays (FIG. 1E).

### 2. miR92a-3p, miR320a and miR-320b Regulate Tau Expression in a Neuroblastoma Cell Line

We next assessed the functional role of the nine validated miRNAs in regulating Tau expression levels. Western blot experiments were carried out upon transient transfection of the miRNAs-overexpressing plasmids in Kelly neuroblastoma cells (Supplementary FIG. 1). They showed that miR-92a-3p, miR-320a, miR-320b, and let7e-5p significatively reduce Tau levels to 75%, 84%, 83% and 87%, respectively, compared to miR-181a as a control (FIG. 2A). No significant variation in Tau levels was observed upon overexpression of miR-190b, miR-320c, miR-320d or miR-1260a, while let7a-5p overexpression unexpectedly significantly increased Tau levels. In this experiment miR-181a was chosen as a negative control since it was not enriched in the capture samples in the above described miR-CATCH experiment, neither was predicted to bind to MAPT mRNA by algorithms commonly used for miRNA targets predictions (e.g. miRDB—www.miRDB.org—or TargetProfiler—mirna.imbb.forth.gr/Targetprofiler).

Kelly cells were then transfected with LNA-based miRNA inhibitors for miR-320a, miR-320b and miR-92a-3p, which induced a significant increase in Tau levels, to 200%, 173%, 144%, respectively, compared to Control LNA (FIG. 2B), as assessed by Western Blots.

To assess the most probable target sites for miR-92a-3p, miR-320a and miR-320b in MAPT mRNA, the miRNA sequences were individually in silico hybridized to the best fitting part of the mRNA sequence, and their hybridization minimum free energy (mfe) was computed, using RNAHybrid (FIG. 2C).

Predicted binding sites of miR-320a and miR-320b (region 6353-6373, part of the 3′UTR) overlapped and their mfe was the same, as expected for the two miRNAs, which differ only by one nucleotide at their 3′ end. MiR-92a-3p was also predicted to bind to the MAPT 3′UTR, in region 2890-3011.

### 3. Plasma miR-92a-3p, miR-320a and miR-320b Levels Differ Between Patients and Healthy Controls

After demonstrating that miR-92a-3p, miR-320a and miR-320b regulate MAPT, we investigated their differential expression in plasma samples of patients with AD and FTD and their possible roles as diagnostic biomarkers.

We enrolled a population of 118 subjects, of which 43 healthy controls, 25 females and 18 males (mean age 72.7±7.4), 33 patients with AD, 19 females and 14 males (mean age 69.1±10.4, mean MMSE score 17.3±4.9), 42 with FTD, 28 women and 14 men (mean age 71.1±8.7, mean MMSE score 21.5±5.9), of which 17 subjects displayed the behavioural variant and 17 the semantic variant. All patients were sporadic and no mutations were found in the genes most involved in AD and FTD: APP, PSEN1, PSEN2, MAPT, GRN, and C9ORF72. All the characteristics of the enrolled subjects was summarized in Table 1.

Quantitative assays were performed by qRT-PCR (FIG. 3), using miR-93a-5p as endogenous control (Grasso et al., Neurobiol Aging 2019; 84:240). Reduced levels of miR-92a-3p were found in both groups of patients compared to healthy controls (FTD 0.83±0.1 versus HC 1.36±0.2, p=0.027; AD 0.78±0.2 versus HC 1.36±0.2, p=0.037). On the contrary, levels of miR-320a did not show differences between patients and controls, but patients with AD had significantly lower levels than patients with FTD (AD 0.81±0.2 versus FTD 1.45±0.1, p=0.009). On the other hand, miR-320b was doubled in both groups of patients respect controls (FTD 0.17±0.02 versus HC 0.09±0.008, p<0.0001; AD 0.17±0.05 versus HC 0.09±0.008, p=0.05).

### 4. Circulating miR-92a-3p, miR-320a and miR-320b Show Sex-Related Differences

As shown in FIG. 3, the downregulation of miR-92a-3p observed in all patients related to controls was confirmed only in women (females: FTD 0.82±0.20 versus HC 1.50±0.20, p=0.02; AD 0.76±0.30 versus HC 1.50±0.20, p=0.045). The analysis of miR-320a by sexes confirmed a lower concentration in AD than FTD in males. Moreover, a significant difference in AD versus healthy controls was also find in men (males: AD 0.44±0.10 versus HC 1.41±0.20, p=0.001; AD 0.44±0.10 versus FTD 1.63±0.20, p=0.0003). On the contrary, we did not observe any inter-group difference in females for miR-320a. Finally, the increase of miR-320b in patients was confirmed only in FTD compared with controls after sex-based stratification (males: FTD 0.17±0.02 versus HC 0.10±0.01, p=0.002; females: FTD 0.17±0.02 versus HC 0.08±0.01, p=0.001).

We then focused on the analysis of FTD sub-phenotypes, but we did not observe any difference among linguistic and behavioural variants. Moreover, possible correlations between miRNAs concentration and age at onset or MMSE score were studied, not finding any significant results.

### 5. ROC Showed a Good Diagnostic Accuracy for all Three miRNAs Analysed

Lastly, to determine the diagnostic accuracy of the three miRNAs as possible biomarkers, we built

ROC curves and calculated the AUC (FIG. 4). The miR-92a-3p distinguished healthy controls from AD patients with AUC=0.76 (p=0.0002) and best accuracy of (60±5)% and 63% sensitivity, both in females (AUC=0.77; p=0.003; (62±6)% accuracy; 52% sensitivity) and in males (AUC=0.75; p=0.02; (50±2)% accuracy; 93% sensitivity). It also identifies controls from FTD patients with AUC=0.69 (p=0.006; (60±5)% accuracy; 26% sensitivity) that reached a value of 0.72 in females (p=0.007; (65±6)% accuracy; 38% sensitivity), while it was not significant in males (AUC=0.59; p=0.41; (52±8)% accuracy; 29% sensitivity). The same miRNA did not have a good diagnostic accuracy for discrimination of the two patients' groups nor in the whole population neither after sex-based stratification. Regarding miR-320a, we observed that it discriminates controls from AD with AUC=0.73 (p=0.001; (61±4)% accuracy; 84% sensitivity) that reached the value of 0.86 in males after sex-stratification (p=0.001; (80±6)% accuracy; 63% sensitivity), while its ability to recognize FTD from controls in females was not significant. Interestingly, this miRNA distinguishes FTD and AD with AUC=0.76 (p=0.0001; (64±4)% accuracy; 78% sensitivity), reaching 0.88 for males (p=0.0008; (77±6)% accuracy; 93% sensitivity) and 69% for females (p=0.03; (61±5)% accuracy; 89% sensitivity). Finally, miR-320b showed a good diagnostic feature in FTD/HC discrimination with an AUC=0.78 (p<0.0001; (74±4)% accuracy; 92% sensitivity) in the whole population, AUC=0.84 (p=0.003; (74±7)% accuracy; 87% sensitivity) in males and AUC=0.75 (p=0.003; (75±4)% accuracy; 96% sensitivity) in females, but not in differentiating AD from controls. Furthermore, it was able to differentiate between FTD and AD, but with a low accuracy in whole population (AUC=0.66; p=0.02; (61±4)% accuracy; 97% sensitivity), and not significant both in males and in females.

### 6. The Combined Measure of miR-92a-3p, miR-320a and miR-320b Partially Improves Diagnostic Accuracy

As a further step, several different implementations of a Bayesian classifier specifically designed on miRNAs expression were tried. The classifier relies on the ΔCt expressions of miR-92a-3p, miR-320a, miR-320b referred, as above, to the normalizer miR-93a-5p. A score is built as a linear combination of the three-ΔCt expressions: Score=ΔCt92a+a*ΔCt320a+b*ΔCt320b, where each one of the two coefficients a and b is varied within the range [−50, 50] with step 0.1. The goal is to find the linear combination that maximizes the diagnostic accuracy. This last parameter is evaluated by assuming the set of scores corresponding to each one of the two, a priori determined, diagnostic classes to be normally distributed. Under this hypothesis, the accuracy as well as the threshold to discriminate between the two classes can be analytically calculated. The normality of each optimal score, namely the score corresponding, for each diagnostic case, to the linear combination that maximizes accuracy, was checked via the Shapiro-Wilk normality test.

The sum of miR-92a-3p, miR-320a and miR-320b to discriminate AD and controls provided a Bayesian classifier with a p value<0.001, an accuracy of (73±4)% and 55% sensitivity. After sex-stratification, the sum of the three miRNAs to distinguish AD and controls showed (68±6)% and (86±5)% of accuracy with p value 0.02 and 0.001 and sensitivity of 44% and 69%, respectively in females and males. Related to FTD vs HC, the combination of all miRNAs showed an accuracy of (73±5)% in the whole population (p<0.001 and 78% sensitivity), of (77±6)% in females (p<0.001 and 80% sensitivity), and (71±7)% in males (p=0.02 and 75% sensitivity). Moreover, when we evaluated the capacity to discriminate AD and FTD, we obtained an accuracy of (69±5)%, (71±6)%, and (85±5)% respectively in the whole population, females, and males, with p<0.001 and 91% sensitivity, p=0.003 and 70% sensitivity, and p=0.001 and 92% sensitivity, respectively.

Some candidates as diagnostic biomarkers of AD and FTD in plasma samples were found. Firstly, miR-CATCH technique was used to find all those miRNAs that interact with the MAPT transcript. Ten miRNAs have been selected to verify their effect on Tau levels. MiR-92a-3p, miR-320a and miR-320b were selected to analyse their levels in plasma samples of patients with FTD and AD respect to HCs. The miR-92a-3p is under-expressed in studied pathological conditions compared to controls, a difference that we found even after stratification by gender in females. Moreover, miR-320a appears to be upregulated in FTD patients in comparison to AD also in men when we stratified by sex. It is therefore useful for differential diagnosis. Respect to HC, the only difference is observed in men with AD who have reduced levels of miR-320a. Instead, miR-320b is up-regulated in both dementias, ROC curve analysis showed that miR-92a-3p and miR-320a are good biomarkers to discriminate AD from HC, while miR-320b to discriminate FTD from HC particularly in males. Combining three miRNAs improves the accuracy only in females, particularly for differential diagnosis (FTD vs AD) and to distinguish FTD from HC.

The miRNAs SEQ ID_1, 2 and 3, perform well in the differential diagnosis of FTD and AD, that is, they distinguish AD from FTD and FTD from HCs, two tasks that presently no biomarker performs.

Even if accuracy of existing CSF AD biomarkers is higher than our plasmatic miRNAs for AD diagnosis, plasmatic miRNAs are obtained by less invasive and easy-to-obtain samples, also useful to monitor the efficacy of a drug by serial analysis during the follow-up and available non only in specialized centers as for PET and CSF analysis.

In conclusion, our results identify miR-92a-3p and miR-320a as good biomarkers to discriminate AD from HC, while miR-320b to discriminate FTD from HC, particularly in males. Combining three miRNAs improves the accuracy in females, particularly for differential diagnosis (FTD vs AD) and to distinguish FTD from HC.

### 7. Experimental Section

### 7.1 Cell Cultures

Human neuroblastoma Kelly cell line was cultivated in RPMI-1640 medium (Gibco®, Life Technologies) supplemented with 2 mM L-Glutamine, Penicillin/Streptomycin and 10% Fetal Bovine Serum (FBS). All cell cultures were maintained at 37° C. in a humidified atmosphere of 5% C02.

7.2 microRNA-Capture Affinity Technology (miR-CATCH)

The miR-CATCH is based on the pull down of the mRNA of interest together with their naturally bound miRNAs after reversible crosslinking (FIG. 1A). The method was performed as described [18].

### 7.3 DNA Capture Oligonucleotides Construction

The MFold web server (http://mfold.rna.albany.edu/?q=mfold) was used to model the secondary structure of MAPT mRNA. The thermodynamically most stable structures were analysed with UGENE (http://ugene.unipro.ru/) to identify the single stranded regions. A specific 5′ biotinylated DNA capture oligonucleotide (5′-GGG TGG TGT CTT TGG AGC GG-3′) was designed against region 1855-1875 of MAPT transcript variant 9 (NCBI NM_001377265.1), region present in all MAPT RNA variants (FIG. 1B). The specificity of the capture oligo was analysed with Basic Local Alignment Search Tool (BLAST, http://blast.ncbi.nlm.nih.gov/Blast.cgi), showing a 17 bp complementarity to the Homo sapiens visual system homeobox 1 (VSX1) transcript variant 2 mRNA. BLAST A scrambled DNA 5′ biotinylated oligonucleotide (5′-ATA TAT TAG ATT GCG TAT AAT TAG G-3′) was designed as non-specific control for the miR-CATCH. This control oligo does not recognise any human transcript, according to the BLAST analysis performed.

### 7.4 Cross-Linking, Hybridization and Co-Precipitation of Complexes

Kelly neuroblastoma cells were crosslinked with 2% paraformaldehyde. After several washes with ice-cold DPBS (Dulbecco's Phosphate Buffered Saline), cells were lysed and homogenized with glass beads. In parallel, biotinylated capture or scramble oligonucleotides were immobilised on MyOne streptavidin magnetic beads (Dynabeads Magnetic Separation Technology—Thermo Fisher Scientific). Cell lysates were incubated with these beads. After several washes, the beads were incubated at 60° C. for 5 minutes to reverse the interaction between the biotin-labelled DNA:mRNA:miRNA complexes and the magnetic beads. Cross-linked nucleic acids and proteins were incubated for 45 minutes at 70° C. to reverse the cross-linkages. Samples were subsequently used for the following validations.

7.5 mRNAs and miRNAs Quantification

For the quantification of MAPT transcripts from total RNA, cDNA was synthesized by retrotranscription using RevertAid First Strand cDNA Synthesis Kit (Thermo Scientific®) with oligo (dT) primers, according to the manufacturer's protocol. Real-Time PCR was performed using Kapa SYBR fast qPCR master mix (Kapa Biosystem®) and primers MAPT_Forward (5′-ACATCCATCATAAACCAGGAGGT-3′) and MAPT Reverse (5′-TGTCTTGGCTTTGGCGTTCT-3′). PCR reaction (10 μl) contains 0.3 μM of each primer, 5 μl of master mix and 10 ng of cDNA. Then, the levels of mRNAs measured in capture compared to the levels obtained in scramble samples were analysed by the 2-□Ct method.

The miRNome pull-down in the capture and scramble samples of the miR-CATCH experiments were analysed by reverse-transcription and Real Time PCR (qRT-PCR) using microRNA Ready-to-Use PCR Human Panels (Exiqon). This analysis was performed on 3 independent captures and 3 scramble samples. For each capture or scramble sample two panels covering 752 human miRNAs were investigated. Data were analysed with Genex software. First inter-plate calibration was performed to compare the results of the first and the second Exiqon panel by using UniSp3 IPC samples present in the plates. Then, N/A values (not expressed miRNA) were substituted with a Ct of 40. Samples with at least 66% of total values were selected, resulting in measurable data for 272 miRNAs. The normalization was performed by the global mean of all miRNAs' Cts and ΔΔCt for each selected miRNA. was obtained by using ΔCt of scramble samples, as controls. The validation of selected miRNAs was performed by TaqMan® microRNA Assay (Applied Biosystems).

7.6 microRNA Overexpression

The plasmids over-expressing nine miRNAs (let-7a-5p, let-7e-5p, miR-190b, miR-1260a, miR-320a, miR-320b, miR-320c, miR-320d, miR-92a-3p) and miR-181a as a negative control have been generated by cloning a fragment containing the precursor region of miRNAs (pre-miRNA) in the psiUx plasmid, using BglII and XhoI or BglII and KpnI restriction sites. PCR amplification of the fragments was performed on human genomic DNA (#G1471 Promega) with PCR primers detailed in Supplementary Table 1. Plasmids for miR-1260a and miR-181a were available from previous literature works.

SUPPLEMENTARY TABLE 1

| SEQ. | | |
|---|---|---|
| ID | Primer name | Primer sequence 5′->3′ |
| 4 | Forward hsa-miR- | ACTGGGTACCGTGGCCTGCTATTTC |
| | 92a-1-3p | |
| 5 | Reverse hsa-miR- | ACTGCTCGAGCTGACACGCAACC |
| | 92a-1-3p | |
| 6 | Forward hsa-miR- | AACTGGGTACCACGAGGACAAGAAAAGG |
| | 190b | |
| 7 | Reverse hsa-miR- | |
| | 190b | |
| 8 | Forward hsa-miR- | ACTGGGTACCCTACCCCGAAGCTC |
| | 320a | |
| 9 | Reverse hsa-miR- | ACTGCTCGAGCCTCTCAGCTCCTC |
| | 320a | |
| 10 | Forward hsa-miR- | ACTGGGTACCGTCCTAGCACCTTTGTT |
| | 320b | |
| 11 | Reverse hsa-miR- | AACTGCTCGAGGCCATGCTTGATTCCA |
| | 320b | |
| 12 | Forward hsa-miR- | ACTGGGTACCGCCTTTGACCTGTTGA |
| | 320c | |
| 13 | Reverse hsa-miR- | ACTGCTCGAGGGAAATTCCCTTAGTCTG |
| | 320c | |
| 14 | Forward hsa-miR- | ACTGGGTACCCTCATAAAGCCTCTGAATATTGG |
| | 320d | |
| 15 | Reverse hsa-miR- | ACTGCTCGAGAGCGAGGCTCTGTCTCAATTA |
| | 320d | |
| 16 | Forward hsa-let- | ACTGGGTACCGCGGATTCAGATAACC |
| | 7a-5p | |
| 17 | Reverse hsa-let- | ACTGCTCGAGCCCCATCCAGTGTA |
| | 7a-5p | |
| 18 | Forward hsa-let- | ACTGGGTACCACCCGTAGAACCGAC |
| | 7e-5p | |
| 19 | Reverse hsa-let- | ACTGCTCGAGAGGGAGTCAGAGGATC |
| | 7e-5p | |

For overexpression experiments, 5×105 cells per well were seeded in 24-well dishes and transfected with miRNA-overexpressing plasmids and Lipofectamine3000® (Life Technologies) in a 3:1 ratio. After 48 hours proteins were extracted and analysed.

7.7 microRNA Inhibition

Kelly cells were transfected with miRCURY LNA microRNA Inhibitors (Exiqon) for hsa-miR-92a-1-3p, hsa-miR-320a and hsa-miR-320b and negative Control A (Exiqon).

For inhibition experiments, Kelly cells were transfected at 50-60% confluence with Lipofectamine® 3000 without P3000 reagent and 12.5 mM/well of miRNA LNA inhibitors. After 48 hours proteins were extracted and analysed.

### 7.8 Western Blot

20 μg of proteins were separated by 10% SDS-polyacrylamide gel electrophoresis (SDS-PAGE) and transferred on nitrocellulose membrane by using the iBlot® Dry Blotting System (Life Technologies) at 20V for 7 minutes. Blots were first blocked with 5% Bovine Serum Albumin (BSA) in PBST (PBS+1% Tween 20). Membrane was incubated with a dilute solution of primary rabbit polyclonal anti-Tau Dako and GAPDH (Agilent Technologies® A0024, 1:1000) in 1% powdered milk at room temperature for 1 hour or over-night at 4° C. and then for 1 hour at room temperature with a diluted solution of secondary fluorescent antibody (Abcam®) in 1% of BSA. Membranes were scanned with the LI-COR Odyssey Infrared Imaging System according to the manufacturer's instructions. Densitometric analysis was performed using Image Studio® and results were normalized by housekeeping GAPDH.

### 7.9 Subject's Recruitment

Patients were recruited from Memory Clinic of “Sapienza” University (Rome, Italy) and from Alzheimer's Unit of IRCCS Neuromed (Pozzilli, Italy). FTD was diagnosed according to current international diagnostic criteria [21, 22](Neary et al, 1998; Rascovsky et al, 2011), while the AD diagnosis followed DSM-IV and NINCDS-ADRDA criteria [23](McKham et al, 1984). Moreover, all patients underwent standard evaluations including 1) a detailed clinical history recorded from the patients and/or caregivers; 2) an extensive physical exam; 3) neurological examination; 4) an extensive neuropsychological testing including the Mini-Mental State Examination (MMSE); 5) laboratory tests to exclude secondary causes of dementia; 6) brain imaging (magnetic resonance imaging or computerized tomography); and 7) in most cases, 99mTc-HMPAO SPECT or FDG PET scan. All patients were screened for possible mutations in APP, PSEN1, PSEN2, MAPT, GRN, and C9ORF72 genes. Controls were healthy volunteers cognitively normal enrolled among patients' partners or caregivers. All of them were subjected to a neurological assessment to evaluate cognitive and functional state. The study was approved by ethical committees of both institutes and all the enrolled subjects gave an informed consent.

7.10 Samples Collection and miRNA Quantification

Whole blood was collected from each subject in tubes with EDTA and centrifuged at 2500 rpm, 4° C. for 15 minutes to obtain plasma aliquoted in 250 μL and stored at −80° C. miRNA extraction was performed by the miRNeasy Serum/Plasma Kit (Qiagen), and reverse transcription into cDNA by miScript II RT Kit (Qiagen). cDNA obtained was diluted 1:10 and quantified by qRT-PCR in according to miRCURY LNA SYBR green PCR Kit (Qiagen) manufacturer's protocol. A critical point in this methodology was to avoid cellular contamination and haemolysis of plasma samples. To check for possible haemolysis contamination of samples, we compared the levels of two miRNAs: hsa-miR-23a-3p, unaffected by haemolysis, and hsa-miR-451a, highly expressed in red blood cells. As reference, we used hsa-miR-93a-5p chosen as the best endogenous control in a previous publication. Data of qRT-PCR were expressed as 2-DeltaCt.

### 7.11 Statistical Analysis

The DeltaCt values of miRNA levels was expressed as mean±standard error. We applied the T-Student test to obtain the p values and compare differential expression between two groups (p≤0.05). To compare multiple groups, we used ANOVA test and Bonferroni Post-hoc, while Pearson test was used for the correlation analysis. Moreover, we built Receiver Operating Curves (ROCs) and calculated the AUC (Area Under Curve) for each miRNA and combinations of them, to obtain information about biomarkers' diagnostic accuracy.

miRNA Sequences

MirRBase database is a public searchable database of published MicroRNA (miRNA) sequences and annotation provided by University of Manchester and available on the following Web page: https://www.mirbase.org/.

The MiRBase database version is version 22.1.
RNA Seq ID_1: miR-320b
complete name: hsa-miR-320b
entry: MIMAT0005792
https://www.mirbase.org/cgi-bin/mature.pl?mature acc=MIMAT0005792
>hsa-miR-320b MIMAT0005792

AAAAGCUGGGUUGAGAGGGCAA.

RNA Seq. ID_2: miR-320a
complete name: hsa-miR-320a-3p
entry: MIMAT0000510
https://www.mirbase.org/cgi-bin/mature.pl?mature acc=MIMAT0000510
>hsa-miR-320a-3p MIMAT0000510

AAAAGCUGGGUUGAGAGGGCGA.

RNA Seq. ID_3: miR-92a-3p
complete name: hsa-miR-92a-3p
entry: MIMAT0000092
https://www.mirbase.org/cgi-bin/mature.pl?acc=MIMAT0000092
>hsa-miR-92a-3p MIMAT0000092

UAUUGCACUUGUCCCGGCCUGU.

The following are objects of the invention:
An in vitro diagnostic method for diagnosing and/or for distinguishing by diagnosis Alzheimer's disease (AD) and Frontotemporal Dementia (FTD), and/or for measuring therapeutic effect of a treatment of FTD or AD, comprising the quantitative analysis of at least one RNA sequence selected among the group consisting of RNA Seq. ID_1, RNA Seq ID._2 and RNA Seq. ID_3, or a combination thereof, or a combination of at least one RNA sequence selected among the group consisting of RNA Seq. ID_1, RNA Seq ID_2 and RNA Seq. ID_3 with at least another biomarker, in a human fluid.
A method for selecting subjects for enrollment in a clinical trial involving treatment of tauopathies, the method comprising: determining by diagnosis according to the method of any one of the claims from 1 to 9 the subject as being suitable for the clinical trial.
A diagnostic device or diagnostic kit configured for carrying out quantitative analysis of at least one RNA sequence selected among the group consisting of RNA Seq. ID_1, RNA Seq. ID_2 and RNA Seq. ID_3, or a combination thereof, or a combination of at least one RNA sequence selected among the group consisting of RNA Seq. ID_1, RNA Seq ID_2 and RNA Seq. ID_3 with at least another biomarker, in a human fluid,
wherein said diagnostic device or diagnostic kit comprises a toolkit configured for the identification and quantification of at least one RNA sequence selected among the group consisting of RNA Seq. ID_1, RNA Seq ID_2 and RNA Seq. ID_3, or a combination thereof, or a combination of at least one RNA sequence selected among the group consisting of RNA Seq. ID_1, RNA Seq. ID_2 and RNA Seq. ID_3 with at least another biomarker, in a human fluid.
A RNA sequence selected among the group consisting of Seq. ID_1, RNA Seq. ID_2 and RNA Seq. ID_3, or a combination thereof, or a combination of at least one RNA sequence selected among the group consisting of RNA Seq. ID_1, RNA Seq. ID_2 and RNA Seq. ID_3 with at least another biomarker, for use in a diagnostic method, wherein said diagnostic method diagnoses and/or distinguishes by analysis Alzheimer's disease (AD), Frontotemporal Dementia (FTD), other tauopathies or wherein said diagnostic method measures therapeutic effect of a treatment of FTD, AD or other tauopathies.
An RNA sequence selected among the group consisting of seq. ID_1, RNA Seq. ID_2 and RNA Seq. ID_3, or a combination thereof, or a combination of at least one RNA sequence selected among the group consisting of RNA Seq. ID_1, RNA Seq. ID_2 and RNA Seq. ID_3 with at least another biomarker, for use in a method of treatment of Alzheimer's disease, Frontotemporal Dementia (FTD), or other tauopathies.
Pharmaceutical composition comprising at least an RNA sequence selected among the group consisting of seq. ID_1, RNA Seq. ID_2 and RNA Seq. ID_3, or a combination thereof, for use in a method of treatment of Alzheimer's disease, Frontotemporal Dementia (FTD), or other tauopathies.
Oligonucleotide whose sequence, or part thereof, is complementary to part or entire RNA seq. ID_1, RNA Seq. ID_2 or RNA Seq. ID_3, for use as inhibitor of seq. ID_1, RNA Seq. ID_2 or RNA Seq. ID_3 in a method of treatment of Alzheimer's disease, Frontotemporal Dementia (FTD), or other tauopathies.
Oligonucleotide whose sequence, or part thereof, is complementary to part of or the entire target sequence of RNA seq. ID_1, RNA Seq ID_2 or RNA Seq. ID_3 on MAPT mRNA, for use as inhibitor of the interaction of seq. ID_1, RNA Seq. ID_2 or RNA Seq. ID_3 with MAPT mRNA, in a method of treatment of Alzheimer's disease, Frontotemporal Dementia (FTD), or other tauopathies.

1) An in vitro diagnostic method for diagnosing and/or for distinguishing by diagnosis Alzheimer's disease (AD) and Frontotemporal Dementia (FTD), and/or for measuring therapeutic effect of a treatment of FTD or AD, comprising the quantitative analysis of at least one RNA sequence selected among the group consisting of RNA Seq. ID_1, RNA Seq ID._2 and RNA Seq. ID_3, or a combination thereof, or a combination of at least one RNA sequence selected among the group consisting of RNA Seq. ID_1, RNA Seq ID_2 and RNA Seq. ID_3 with at least another biomarker, in a human fluid.

2) An in vitro diagnostic method according to claim 1, wherein the quantitative analysis is performed by PCR.

3) An in vitro diagnostic method according to claim 1, wherein the human fluid is blood or derivatives thereof.

4) An in vitro diagnostic method according to claim 3, wherein said method comprises the following steps:
a) blood sample collection,
b) isolation of plasma or serum from blood,
c) optionally, RNA extraction,
d) detection of at least one RNA sequence selected among the group consisting of Seq. ID_1, RNA Seq. ID._2 and RNA Seq. ID_3.

5) An in vitro diagnostic method according to claim 1, wherein the RNA sequence is RNA Seq. ID_1.

6) An in vitro diagnostic method according to claim 1, wherein the RNA sequence is RNA Seq. ID_2.

7) An in vitro diagnostic method according to claim 1, wherein the RNA sequence is RNA Seq. ID_3.

8) An in vitro diagnostic method according to claim 1, comprising the quantitative analysis of two RNA sequences being RNA Seq. ID_2 and RNA Seq. ID_3.

9) An in vitro diagnostic method according to claim 1, comprising the quantitative analysis of three RNA sequences being RNA Seq. ID_1, RNA Seq. ID_2 and RNA Seq. ID_3.

10) A method for selecting subjects for enrollment in a clinical trial involving treatment of tauopathies, the method comprising: determining by diagnosis according to the method of claim 1 the subject as being suitable for the clinical trial.

11) A diagnostic device or diagnostic kit configured for carrying out quantitative analysis of at least one RNA sequence selected among the group consisting of RNA Seq. ID_1, RNA Seq. ID_2 and RNA Seq. ID_3, or a combination thereof, or a combination of at least one RNA sequence selected among the group consisting of RNA Seq. ID_1, RNA Seq ID_2 and RNA Seq. ID_3 with at least another biomarker, in a human fluid, wherein said diagnostic device or diagnostic kit comprises a toolkit configured for the identification and quantification of at least one RNA sequence selected among the group consisting of RNA Seq. ID_1, RNA Seq ID_2 and RNA Seq. ID_3, or a combination thereof, or a combination of at least one RNA sequence selected among the group consisting of RNA Seq. ID_1, RNA Seq. ID_2 and RNA Seq. ID_3 with at least another biomarker, in a human fluid.

12-20) (canceled)

21) A method of treatment of Alzheimer's disease, Frontotemporal Dementia (FTD), or other tauopathies comprising using an RNA sequence selected among the group consisting of seq. ID_1, RNA Seq. ID_2 and RNA Seq. ID_3, or a combination thereof, or a combination of at least one RNA sequence selected among the group consisting of RNA Seq. ID_1, RNA Seq. ID_2 and RNA Seq. ID_3 with at least another biomarker.

22) The method according to claim 21, wherein the method of treatment comprises administrating a therapeutically effective amount of said RNA sequence.

23) A method of treatment of Alzheimer's disease, Frontotemporal Dementia (FTD), or other tauopathies comprising using a pharmaceutical composition comprising at least an RNA sequence selected among the group consisting of seq. ID_1, RNA Seq. ID_2 and RNA Seq. ID_3, or a combination thereof.

24) The method according to claim 23, further comprising using at least another therapeutical compound or active pharmaceutical compound.

25) A method of treatment of Alzheimer's disease, Frontotemporal Dementia (FTD), or other tauopathies comprising using an oligonucleotide whose sequence, or part thereof, is complementary to part or entire RNA seq. ID_1, RNA Seq. ID_2 or RNA Seq. ID_3, as inhibitor of seq. ID_1, RNA Seq. ID_2 or RNA Seq. ID_3.

26) A method of treatment of Alzheimer's disease, Frontotemporal Dementia (FTD), or other tauopathies comprising using an oligonucleotide whose sequence, or part thereof, is complementary to part of or the entire target sequence of RNA seq. ID_1, RNA Seq ID_2 or RNA Seq. ID_3 on MAPT mRNA, as inhibitor of the interaction of seq. ID_1, RNA Seq. ID_2 or RNA Seq. ID_3 with MAPT mRNA.


SEQUENCE LISTING (19 sequences)

SEQ ID NO: 1 - RNA, 22 nt, synthetic construct
  source 1..22: mol_type = other RNA
aaaagctgggttgagagggcaa

SEQ ID NO: 2 - RNA, 22 nt, synthetic construct
  source 1..22: mol_type = other RNA
aaaagctgggttgagagggcga

SEQ ID NO: 3 - RNA, 22 nt, synthetic construct
  source 1..22: mol_type = other RNA
tattgcacttgtcccggcctgt

SEQ ID NO: 4 - DNA, 25 bp, synthetic construct
  source 1..25: mol_type = other DNA
actgggtaccgtggcctgctatttc

SEQ ID NO: 5 - DNA, 23 bp, synthetic construct
  source 1..23: mol_type = other DNA
actgctcgagctgacacgcaacc

SEQ ID NO: 6 - DNA, 28 bp, synthetic construct
  source 1..28: mol_type = other DNA
aactgggtaccacgaggacaagaaaagg

SEQ ID NO: 7 - DNA, 27 bp, synthetic construct
  source 1..27: mol_type = other DNA
actgctcgagatagttgcccggatgtg

SEQ ID NO: 8 - DNA, 24 bp, synthetic construct
  source 1..24: mol_type = other DNA
actgggtaccctaccccgaagctc

SEQ ID NO: 9 - DNA, 24 bp, synthetic construct
  source 1..24: mol_type = other DNA
actgctcgagcctctcagctcctc

SEQ ID NO: 10 - DNA, 27 bp, synthetic construct
  source 1..27: mol_type = other DNA
actgggtaccgtcctagcacctttgtt

SEQ ID NO: 11 - DNA, 27 bp, synthetic construct
  source 1..27: mol_type = other DNA
aactgctcgaggccatgcttgattcca

SEQ ID NO: 12 - DNA, 26 bp, synthetic construct
  source 1..26: mol_type = other DNA
actgggtaccgcctttgacctgttga

SEQ ID NO: 13 - DNA, 28 bp, synthetic construct
  source 1..28: mol_type = other DNA
actgctcgagggaaattcccttagtctg

SEQ ID NO: 14 - DNA, 33 bp, synthetic construct
  source 1..33: mol_type = other DNA
actgggtaccctcataaagcctctgaatattgg

SEQ ID NO: 15 - DNA, 31 bp, synthetic construct
  source 1..31: mol_type = other DNA
actgctcgagagcgaggctctgtctcaatta

SEQ ID NO: 16 - DNA, 26 bp, synthetic construct
  source 1..26: mol_type = other DNA
actgggtaccgcggattcagataacc

SEQ ID NO: 17 - DNA, 24 bp, synthetic construct
  source 1..24: mol_type = other DNA
actgctcgagccccatccagtgta

SEQ ID NO: 18 - DNA, 25 bp, synthetic construct
  source 1..25: mol_type = other DNA
actgggtaccacccgtagaaccgac

SEQ ID NO: 19 - DNA, 26 bp, synthetic construct
  source 1..26: mol_type = other DNA
actgctcgagagggagtcagaggatc